(19) United States
(12) Patent Application Publication
(10) Pub. No.: US 2012/0123274 A1
(43) Pub. Date: May 17, 2012

(54) ULTRASONIC TRANSDUCER AND METHOD OF MANUFACTURING THE SAME

(76) Inventors: Masako IKEDA, Sunnyvale, CA (US); Takashi OGURA, Osaka (JP)

(21) Appl. No.: 13/358,652

(22) Filed: Jan. 26, 2012

Related U.S. Application Data
(63) Continuation of application No. PCT/JP2011/002883, filed on May 24, 2011.

(30) Foreign Application Priority Data
May 27, 2010 (JP) 2010-122099

(51) Int. Cl. A61B 8/14; B05D 5/00

(52) U.S. Cl. 600/472; 427/2.12

(57) ABSTRACT

An ultrasonic transducer which can attenuate ultrasonic waves emitted toward the rear without increasing a thickness of a backing layer includes: a piezoelectric transducer which emits and receives ultrasonic waves; and the backing layer which is provided in contact with the rear of the piezoelectric transducer and which attenuates the ultrasonic waves emitted in a rear direction from the piezoelectric transducer. The backing layer includes a plurality of acoustic tubes formed in the rear direction from a plane of the backing layer that is in contact with the piezoelectric transducer. Each of the acoustic tubes has a different length based on a principle of superposition of acoustic waves. The acoustic tubes include an acoustic tube which has (i) a portion of the length formed in a direction perpendicular to the rear direction and (ii) the remaining portion of the length formed in a direction parallel to the rear direction.

### CROSS REFERENCE TO RELATED APPLICATIONS

This is a continuation application of PCT Patent Application No. PCT/JP2011/002883 filed on May 24, 2011, designating the United States of America, which is based on and claims priority of Japanese Patent Application No. 2010-122099 filed on May 27, 2010. The entire disclosures of the above-identified applications, including the specifications, drawings and claims are incorporated herein by reference in their entirety.

### BACKGROUND OF THE INVENTION

(1) Field of the Invention

The present invention relates to ultrasonic transducers used for ultrasonic diagnosis and methods of manufacturing the ultrasonic transducers.

(2) Description of the Related Art

FIG. 1 shows an exemplary appearance of an ultrasonic transducer and an ultrasonic diagnostic apparatus. The ultrasonic transducer 70 is connected to the ultrasonic diagnostic apparatus 80 by a cable. The ultrasonic transducer 70 emits ultrasonic waves in the direction of an arrow shown in the drawing, and receives reflection waves which are reflected from a living body and are in the opposite direction of the arrow. The ultrasonic diagnostic apparatus 80 performs image analysis on the reflected waves received by the ultrasonic transducer 70, and displays on a monitor an image of the inside of the living body obtained through the analysis.

When such an ultrasonic transducer 70 emits the ultrasonic waves from a piezoelectric transducer, the ultrasonic waves are emitted not only to the front of a transducer, but also to the rear of the transducer. The following describes an example of a structure of a conventional ultrasonic transducer with reference to a drawing.

FIG. 2 is a cross-sectional view showing a structure of a conventional ultrasonic transducer 90. The conventional ultrasonic transducer 90 in FIG. 2 has the structure in which, from the top, an acoustic lens 93, a matching layer 92, a piezoelectric transducer 91, and a backing layer 94 are stacked. It is to be noted that FIG. 2 schematically illustrates a thickness of each of materials included in the ultrasonic transducer 90.

In typical ultrasonic diagnostic apparatus, the ultrasonic waves emitted from the piezoelectric transducer 91 passes through the matching layer 92 and the acoustic lens 93, and then emitted into a living body. Subsequently, the ultrasonic waves reflected within the living body pass through the same route in the reverse order as the emitted ultrasonic waves passed, and then received back by the piezoelectric transducer 91. Depending on the strength of reception or response time, a received signal is visualized in shading by the ultrasonic diagnostic apparatus.

On the other hand, as described in the beginning, ultrasonic waves having an opposite phase to the phase of ultrasonic waves that are emitted to the front (upper side in FIG. 2) are emitted from the piezoelectric transducer 91 to the rear at the same time. The ultrasonic waves emitted to the rear of the piezoelectric transducer 91 (lower side in FIG. 2) are attenuated by the backing layer 94. However, when the backing layer 94 is formed of a material which does not sufficiently attenuate the ultrasonic waves, the ultrasonic waves reflect within the backing layer 94 and go back toward the piezoelectric transducer 91.

Due to the effect of the reflected waves described above, on the ultrasonic wave signal that is reflected from the living body and received by the ultrasonic transducer 90, noise is superimposed. With this, properties of the ultrasonic diagnostic apparatus are degraded.

Thus, a material having internal loss and distance that can provide sufficient attenuation to the ultrasonic waves emitted to the rear is provided as the backing layer 94 (for example, Patent Reference 1: Japanese Patent No. 3806349).

### SUMMARY OF THE INVENTION

However, the structure disclosed in the Patent Reference 1, that is, a structure in which a material having internal loss and distance that can provide attenuation to the ultrasonic waves is provided as a backing layer poses a problem of increasing the thickness of the backing layer itself.

The present invention has been conceived to solve the above conventional problem, and has as an object to provide an ultrasonic transducer and a manufacturing method of the ultrasonic transducer which can attenuate ultrasonic waves emitted to the rear without increasing the thickness of the backing layer.

In order to achieve the aforementioned object, an ultrasonic transducer according to an aspect of the present invention includes: a transducer which emits and receives ultrasonic waves; and a backing material which is provided in contact with a rear of the transducer and which attenuates the ultrasonic waves emitted in a rear direction from the transducer. The backing material includes a plurality of reflectors formed in the rear direction from a plane of the backing material that is in contact with the transducer. Each of the reflectors has a different length based on a principle of superposition of acoustic waves. The reflectors include a reflector which has (i) a portion of the length formed in a direction perpendicular to the rear direction and (ii) the remaining portion of the length formed in a direction parallel to the rear direction.

With this structure, for example, a reflector having a long length can be formed with a portion of the reflector bent along the length. Thus, it is possible to realize the ultrasonic transducer which can attenuate ultrasonic waves emitted to the rear without increasing the thickness of the backing material.

Furthermore, it is preferable that each of the reflectors have properties of an acoustic tube. Here, it may be that each of the reflectors is formed to have a length that is an integer multiple of a predetermined unit length, and one of neighboring reflectors which has a greater length has a portion of the length bent in a direction perpendicular to the rear direction so as to be formed in the rear direction of another one of the neighboring reflectors having a smaller length, the neighboring reflectors being included in the reflectors.

Furthermore, in order to achieve the aforementioned object, an ultrasonic transducer according to an aspect of the present invention includes: a transducer which emits and receives ultrasonic waves; and a backing material which is provided in contact with a rear of the transducer and which attenuates ultrasonic waves emitted in a rear direction from the transducer. The backing material includes a plurality of reflectors formed in the rear direction from a plane of the backing material that is in contact with the transducer. Each of the reflectors is formed based on a Helmholtz resonator principle.

With the above structure, the reflectors have properties of the resonators. Further, there is an advantageous effect that it is easy to form the reflectors having the above structure.

Thus, it is possible to realize the ultrasonic transducer which can attenuate ultrasonic waves emitted to the rear without increasing the thickness of the backing material.

Furthermore, in order to achieve the aforementioned object, a method of manufacturing an ultrasonic transducer according to an aspect of the present invention is a method of manufacturing an ultrasonic transducer which includes: a transducer which emits and receives ultrasonic waves; and a backing material which (i) is provided in contact with a rear of the transducer, (ii) includes a board and a plurality of reflectors, and (iii) attenuates ultrasonic waves emitted in a rear direction from the transducer. The method includes forming the backing material which includes the reflectors by printing on the board a material with an acoustic impedance different from an acoustic impedance of the board, each of the reflectors having a different length based on a principle of superposition of acoustic waves and being formed in the rear direction from a plane of the backing material that is in contact with the transducer.

With this, it becomes easier to form the ultrasonic transducer which can attenuate the ultrasonic waves emitted to the rear without increasing the thickness of the backing material.

According to the present invention, it is possible to realize the ultrasonic transducer and the manufacturing method of the ultrasonic transducer which can attenuate the ultrasonic waves emitted to the rear without increasing the thickness of the backing layer.

### BRIEF DESCRIPTION OF THE DRAWINGS

These and other objects, advantages and features of the invention will become apparent from the following description thereof taken in conjunction with the accompanying drawings that illustrate a specific embodiment of the invention. In the Drawings:

FIG. 1 shows an exemplary appearance of an ultrasonic transducer and an ultrasonic diagnostic apparatus;

FIG. 2 is a cross-sectional view showing a structure of a conventional ultrasonic transducer;

FIG. 3 is a cross-sectional view showing a structure of an ultrasonic transducer according to Embodiment 1 of the present invention;

FIG. 4 is a cross-sectional view of a backing layer according to Embodiment 2 of the present invention;

FIG. 5 is a cross-sectional view showing an example of an arrangement of acoustic tubes according to Embodiment 2 of the present invention;

FIG. 6 shows change in amplitudes of noise contained in a signal of the case where the backing layer according to Embodiment 2 of the present invention includes acoustic tubes and the case where the backing layer does not include acoustic tubes;

FIG. 7 is a cross-sectional view showing another example of an arrangement of acoustic tubes according to Embodiment 2 of the present invention;

FIG. 8A is a diagram showing an example of a three-dimensional structure of acoustic tubes according to Embodiment 2 of the present invention;

FIG. 8B is a diagram showing an example of the three-dimensional structure of the acoustic tubes according to Embodiment 2 of the present invention;

FIG. 8C is a diagram showing an example of the three-dimensional structure of the acoustic tubes according to Embodiment 2 of the present invention;

FIG. 8D is a diagram showing an example of the three-dimensional structure of the acoustic tubes according to Embodiment 2 of the present invention;

FIG. 9A is a diagram showing another example of a three-dimensional structure of acoustic tubes according to Embodiment 2 of the present invention;

FIG. 9B is a diagram showing another example of the three-dimensional structure of the acoustic tubes according to Embodiment 2 of the present invention;

FIG. 9C is a diagram showing another example of the three-dimensional structure of the acoustic tubes according to Embodiment 2 of the present invention;

FIG. 9D is a diagram showing another example of the three-dimensional structure of the acoustic tubes according to Embodiment 2 of the present invention;

FIG. 10A is a cross-sectional view showing in which direction the plane having openings of the acoustic tubes formed within the backing layer according to Embodiment 2 of the present invention is provided with respect to the piezoelectric transducer;

FIG. 10B is a cross-sectional view showing in which direction the plane having the openings of the acoustic tubes formed within the backing layer according to Embodiment 2 of the present invention is provided with respect to the piezoelectric transducer;

FIG. 11 is a diagram showing a relation between a direction in which one-dimensionally arranged acoustic tubes are formed within the backing layer according to Embodiment 2 of the present invention and a direction in which the piezoelectric transducer is diced;

FIG. 12A is a cross-sectional view showing an example of a structure of an ultrasonic transducer according to Embodiment 3 of the present invention;

FIG. 12B is a cross-sectional view showing another example of a structure of an ultrasonic transducer according to Embodiment 3 of the present invention;

FIG. 13A is a diagram showing an example of an arrangement of a piezoelectric transducer according to Embodiment 3 of the present invention;

FIG. 13B is a diagram showing an example of an arrangement of acoustic tubes with respect to the piezoelectric transducer according to Embodiment 3 of the present invention;

FIG. 14 is a cross-sectional view showing an example of an arrangement of the acoustic tubes shown in FIG. 12A;

FIG. 15 is a cross-sectional view showing an example of an arrangement of the acoustic tubes shown in FIG. 12B which include portions that are bent;

FIG. 16 is a diagram showing an example of printing patterns according to Embodiment 4 of the present invention;

FIG. 17 is a flowchart showing steps for forming a printing pattern using a screen printing according to Embodiment 4 of the present invention;

FIG. 18 is a diagram showing an example of printing patterns according to Embodiment 5 of the present invention;

FIG. 19A is a cross-sectional view showing a structure of an ultrasonic transducer according to Embodiment 6 of the present invention;

FIG. 19B is a diagram schematically showing a resonator, which is an example of a reflector according to Embodiment 6 of the present invention;

FIG. 20 is a perspective view of a backing layer 4f and shows an example of an arrangement of resonators according to Embodiment 6 of the present invention; and

FIG. 21 is a perspective view of a backing layer and shows an example of another arrangement of resonators according to Embodiment 6 of the present invention.

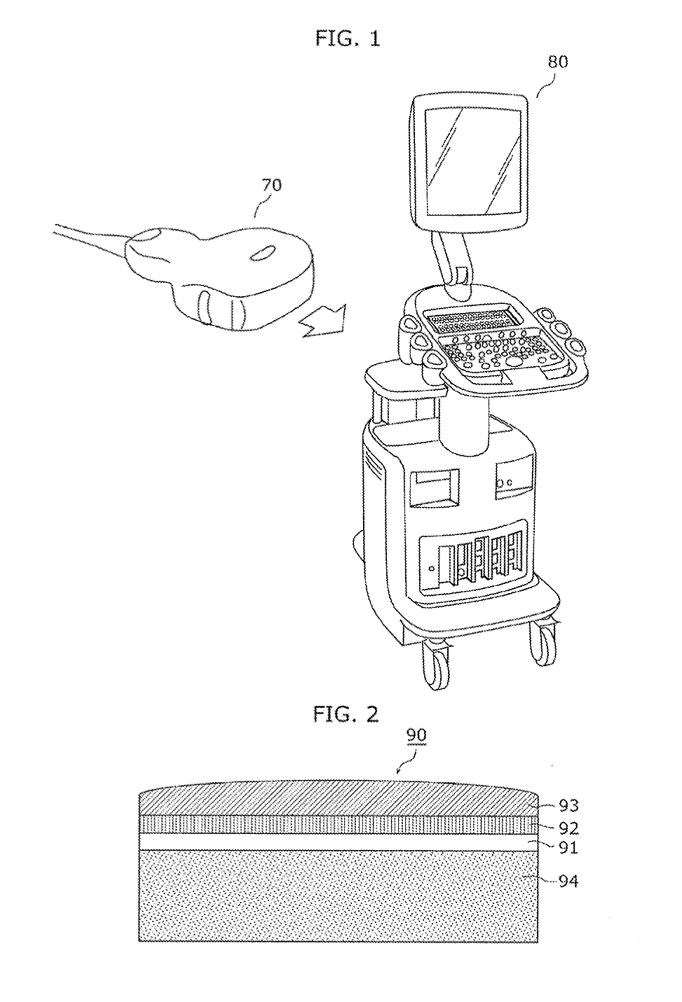

### DESCRIPTION OF THE PREFERRED EMBODIMENTS

Hereinafter, embodiments of the present invention will be described with reference to the drawings.

### Embodiment 1

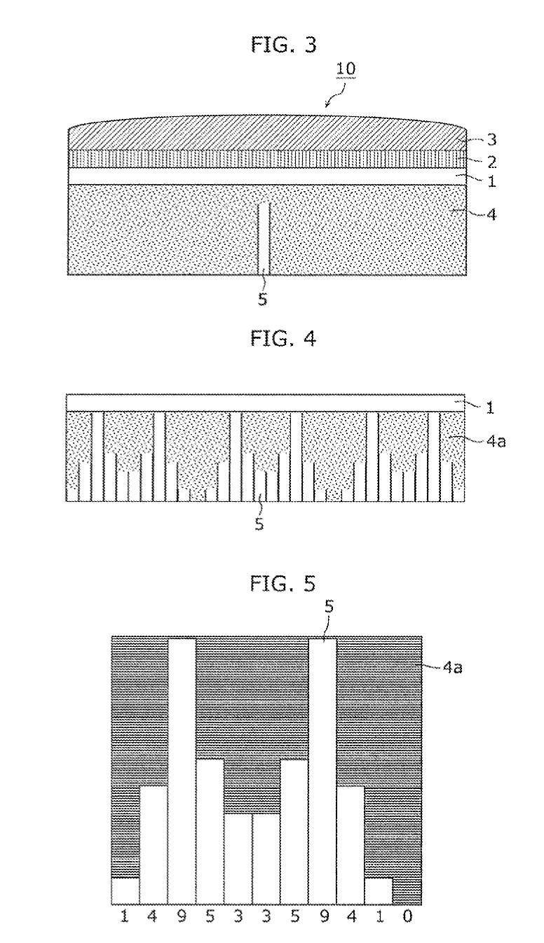

FIG. 3 is a cross-sectional view showing a structure of an ultrasonic transducer according to Embodiment 1 of the present invention. The ultrasonic transducer 10 shown in FIG. 3 includes a piezoelectric transducer 1, a matching layer 2, an acoustic lens 3, and a backing layer 4. In addition, as shown in FIG. 3, the ultrasonic transducer 10 includes an acoustic tube 5 that is formed in the backing layer 4.

The acoustic tube 5 is formed such that its width (w) is sufficiently small compared to the wavelength (λ) of the ultrasonic waves emitted from the piezoelectric transducer 1, and its length (Ln) causes direct waves of the ultrasonic waves and reflected waves of the ultrasonic waves to cancel out each other.

Here, the wavelength λ in the backing layer 4 may be obtained by Equation 1.

$$\begin{matrix}[ Math . 1 ] & \\ λ = \frac{C}{f} & ( Equation 1 )\end{matrix}$$

For example, when it is assumed that the backing layer 4 is made of an epoxy resin and the piezoelectric transducer 1 emits the ultrasonic waves of f=5 MHz. When it is assumed that speed of sound c within the epoxy resin is 5000 m/s, the wavelength of the ultrasonic waves can be obtained as λ=1000 μm.

Then, in this case, when it is assumed that the length Ln of the acoustic tube 5 is 250 μm, a phase of the reflected waves shifts by ¼. This causes cancellation of the ultrasonic waves. Here, the width (w) of the acoustic tube 5 needs to satisfy w<Ln so that a rectilinear propagation of acoustic waves is maintained.

In other words, in the backing layer 4 that is included in the ultrasonic transducer 10, the acoustic tube 5 that has a length based on a principle of superposition of acoustic waves is formed in the rear direction (toward the lower side in the drawing) viewed from a plane of the backing layer 4 that is in contact with the piezoelectric transducer 1. With this, it is possible to attenuate the ultrasonic waves emitted to the rear by the piezoelectric transducer 1 and thereby allow the ultrasonic transducer to receive only the ultrasonic waves reflected from the front side. Thus, it is possible to produce an effect that sensitivity of an ultrasonic wave signal is increased and thus a good image can be obtained with the ultrasonic diagnostic apparatus which includes the ultrasonic transducer 10.

As described above, according to the ultrasonic transducer in Embodiment 1, the acoustic tube 5 is formed in the backing layer 4. With this, it is possible to attenuate the ultrasonic waves without increasing the thickness of the backing layer compared to the case where the material having internal loss and distance that can provide attenuation to the ultrasonic waves is provided as the backing layer.

### Embodiment 2

Although Embodiment 1 has described an example where one acoustic tube is formed in a backing layer, the present invention is not limited to this. Embodiment 2 describes the case where a plurality of acoustic tubes is arranged in the backing layer.

FIG. 4 is a cross-sectional view of the backing layer 4a according to Embodiment 2 of the present invention. Above the backing layer 4a shown in FIG. 4, in addition to a piezoelectric transducer 1, although not illustrated, a matching layer 2 and an acoustic lens 3 are stacked in the same manner as shown in FIG. 3.

As shown in FIG. 4, a plurality of acoustic tubes 5 is arranged in the backing layer 4a. Here, the acoustic tubes 5 have lengths (Ln) based on a principle of superposition of acoustic waves. The lengths (Ln) of the acoustic tubes 5 are arranged according to a defined rule.

Following describes the lengths (Ln) of the acoustic tubes 5.

FIG. 5 is a cross-sectional view of the backing layer 4 showing an example of an arrangement of the acoustic tubes 5 according to Embodiment 2 of the present invention. FIG. 5 shows an example where the acoustic tubes 5 are arranged in the backing layer 4a based on a quadratic residue sequence. Specifically, length (Ln) of each of the acoustic tubes is determined by a one dimensional quadratic residue sequence which satisfies the Equation 2 below.

$$\begin{matrix}[ Math . 2 ] & \\ Ln = \frac{c \cdot {n}^{2} ( mod N )}{2 N ω r} & ( Equation 2 )\end{matrix}$$

Here, c denotes a speed of sound, N denotes a prime number, and n denotes an integer which varies in a range of 0 to (N-1), and ωr denotes any design frequency.

For example, it is assumed that the backing layer 4 is made of an epoxy resin and the speed of sound c within the epoxy resin is 5000 m/s, N=11, and ωr=5 MHz. In this case, each acoustic tube 5 in the backing layer 4 has, with 45.5 μm as a unit length “1”, a length of 1, 4, 9, 5, 3, 3, 5, 9, 4, 1, and 0 respectively.

The acoustic tubes 5 arranged based on the arrangement with the lengths (Ln) which satisfy the above Equation 2 are known to absorb and spread the acoustic waves of broadband because a discontinuity of phase occurs in the vicinity of an opening of each of adjacent acoustic tubes 5. In other words, the reflected waves can be reduced by arranging within the backing layer 4 the acoustic tubes 5 based on the arrangement with the lengths (Ln) which satisfy the above Equation 2.

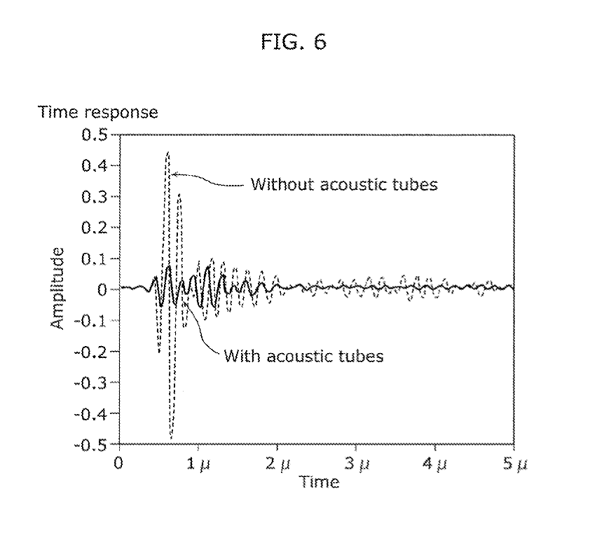

An example of an effect of arranging acoustic tubes 5 based on the arrangement with the lengths (Ln) which satisfy the above Equation 2 is shown in FIG. 6. FIG. 6 shows change in amplitudes of noise contained in a signal of the case where the backing layer according to Embodiment 2 of the present invention includes acoustic tubes and the case where the backing layer does not include acoustic tubes. As shown in FIG. 6, when the backing layer 4a includes acoustic tubes 5 arranged based on the arrangement with the lengths (Ln) which satisfy the above Equation 2, the amplitude of noise is less than the case where the backing layer 4a includes no acoustic tubes. This indicates that the acoustic tubes 5 can absorb and spread the noise.

It is to be noted that the lengths (Ln) of the acoustic tubes 5 are not limited to the lengths arranged based on the quadratic residue sequence. The length (Ln) of each acoustic tube 5 may be arranged based on a primitive root sequence which satisfies Equation 3 below. With this, similar effect can be produced.

$$\begin{matrix}[ Math . 3 ] & \\ Ln = \frac{c \cdot {r}^{n} ( mod N )}{2 ( N - 1 ) ω r} & ( Equation 3 )\end{matrix}$$

Here, c denotes speed of sound, N denotes a prime number, and n denotes an integer which varies in a range of 0 to (N-1), r denotes a primitive root of N, and ωr denotes any design frequency.

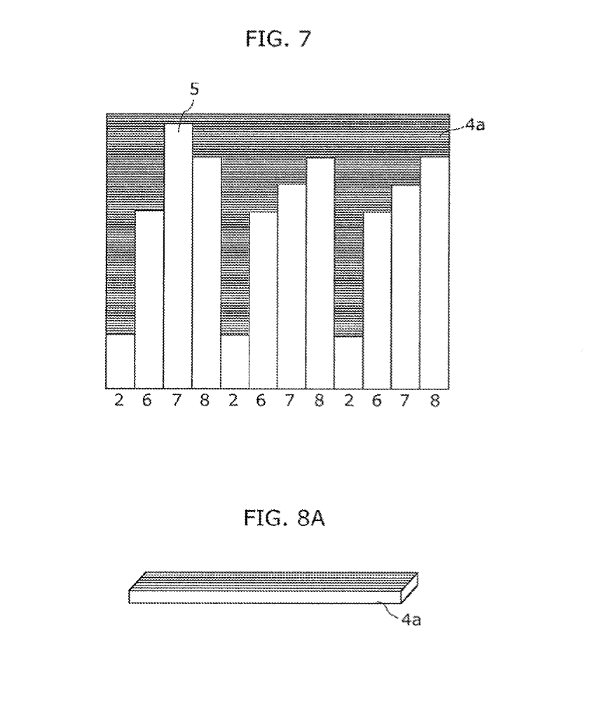

FIG. 7 is a cross-sectional view of the backing layer 4 and shows another example of an arrangement of the acoustic tubes 5 according to Embodiment 2 of the present invention. As an example of the backing layer in which each of the acoustic tubes 5 is arranged based on a primitive root sequence, FIG. 7 shows an arrangement of the acoustic tubes 5 when N=11 and r=2.

Furthermore, the arrangement of the acoustic tubes 5 is not limited to the one-dimensional arrangement shown in FIG. 5 and FIG. 7, and a two-dimensional arrangement may also be used.

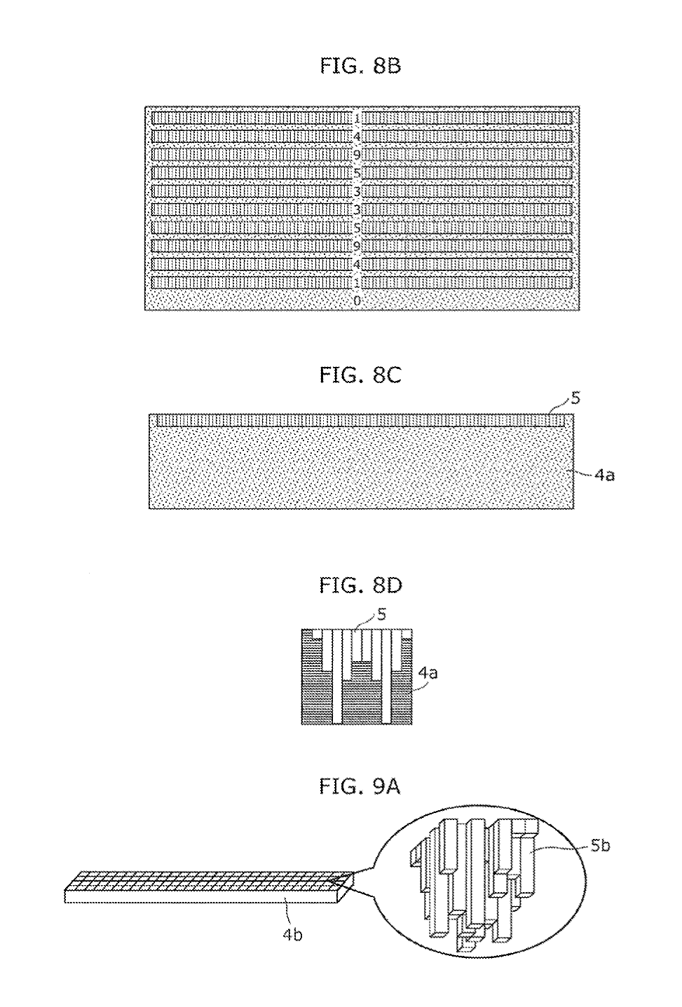

FIG. 8A to FIG. 8D are diagrams which show an example of a three-dimensional structure of the acoustic tubes according to Embodiment 2 of the present invention. FIG. 8A is a perspective view showing the backing layer 4a in which the acoustic tubes 5 are formed based on the one-dimensional arrangement shown in FIG. 5. FIG. 8B to FIG. 8D compose a three-view drawing of FIG. 8A. FIG. 8B is a plan view, FIG. 8C is a front view, and FIG. 8D is a side view. For example, as shown in FIG. 8B, grooves which are parallel in the horizontal direction are formed in the backing layer 4a. The grooves are formed to have depths (lengths of the acoustic tubes) of 1, 4, 9, 5, 3, 3, 5, 9, 4, 1, and 0 in sequence in the vertical direction. As shown in FIG. 8C, a depth of each of the grooves that form the acoustic tubes 5 is uniform. When the backing layer 4a is cut along a plane perpendicular to a longitudinal direction of the grooves, the depth of each groove (length (Ln) of each acoustic tube 5) is arranged in the quadratic residue sequence as shown in FIG. 8D.

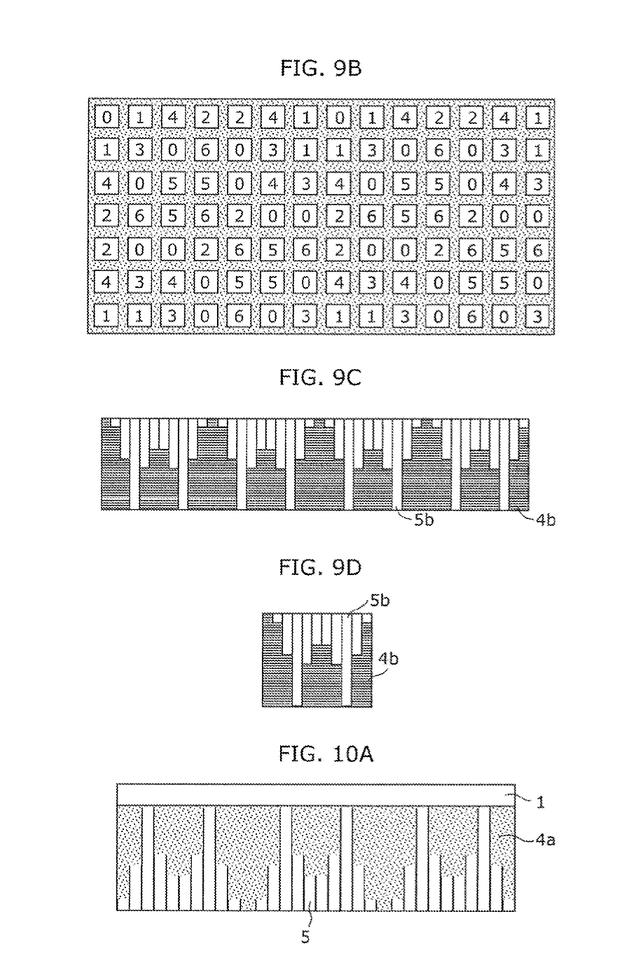

FIG. 9A to FIG. 9D are diagrams which show an example of another three-dimensional structure of the acoustic tubes according to Embodiment 2 of the present invention. FIG. 9A is a perspective view showing the backing layer 4b in which acoustic tubes 5b are formed in two-dimensional arrangement. FIG. 9B to FIG. 9D compose a three-view drawing of FIG. 9A. FIG. 9B is a plan view, FIG. 9C is a front view, and FIG. 9D is a side view. For example, as shown in FIG. 9B, in the backing layer 4b, grooves are formed to have various to depths in two-dimensional directions of horizontal direction and vertical direction. In the case where c=5000 m/s, N=7, and ωr=5 MHz, the grooves are formed to have a depth of an integer multiple of a unit length of 71.5 μm. Furthermore, as shown in FIG. 9C and FIG. 9D, the grooves are arranged such that the depths of the grooves are repeated in a predetermined pattern when viewed from the direction perpendicular to the cross-section as well as when viewed from the direction horizontal to the cross-section.

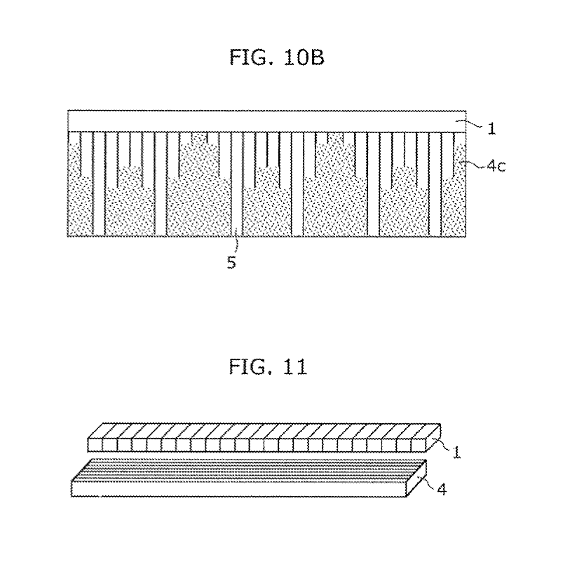

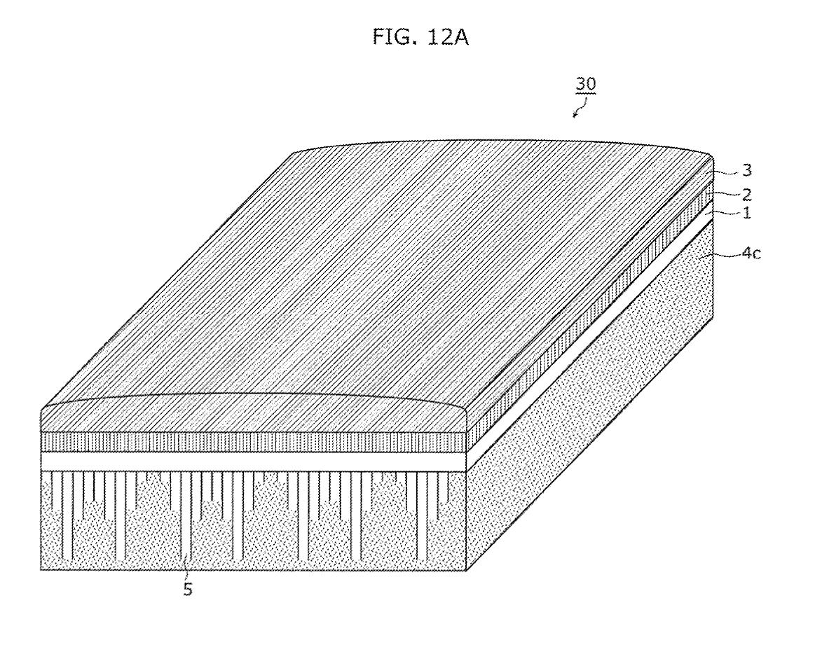

FIG. 10A and FIG. 10B are cross-sectional views showing in which direction the plane having the openings of the acoustic tubes formed within the backing layer according to Embodiment 2 of the present invention is provided with respect to the piezoelectric transducer 1. FIG. 10A shows, in the same manner as in the FIG. 5, an example where the plane of the backing layer 4a without the openings of the acoustic tubes 5 that are formed in the backing layer 4a is provided in contact with the layer of the piezoelectric transducer 1. On the other hand, FIG. 10B shows an example where the plane of the backing layer 4c with the openings of the acoustic tubes 5 is provided in contact with the layer of the piezoelectric transducer 1.

Stated differently, to cancel out the reflected waves, which are the ultrasonic waves that return after having reflected off the end of the backing layer, the plane having the openings of the acoustic tubes 5 may be formed at either side with respect to the piezoelectric transducer 1 as shown in FIG. 10A and FIG. 10B.

FIG. 11 is a diagram showing a relation between a direction in which one-dimensionally arranged acoustic tubes are formed within the backing layer according to Embodiment 2 of the present invention and a direction in which the piezoelectric transducer is diced. As shown in the drawing, when the acoustic tubes 5 are formed within the backing layer 4 in a one-dimensional arrangement, it is preferable that the acoustic tubes 5 be formed such that a direction of dice cutting of the piezoelectric transducer 1 and a longitudinal direction of the grooves of the acoustic tubes 5 are at right angles to each other. With this, a larger number of the acoustic tubes of different lengths act on the piezoelectric transducer of one channel. Thus, the reflected waves can be reduced more effectively within the backing layer 4.

As described above, according to the ultrasonic transducer in Embodiment 2, the acoustic tubes are arranged in the backing layer. With this, it is possible to attenuate the ultrasonic waves without increasing the thickness of the backing layer compared to the case where the material having internal loss and distance that can provide attenuation to the ultrasonic waves is provided as the backing layer.

### Embodiment 3

Embodiment 1 and Embodiment 2 have described an example where one or more acoustic tubes are arranged in a backing layer. However, the present invention is not limited to this. It is sufficient that reflectors corresponding to the acoustic tubes are arranged in the backing layer. Embodiment 3 describes the case where the reflectors have properties of the acoustic tubes and serve as acoustic tubes 5.

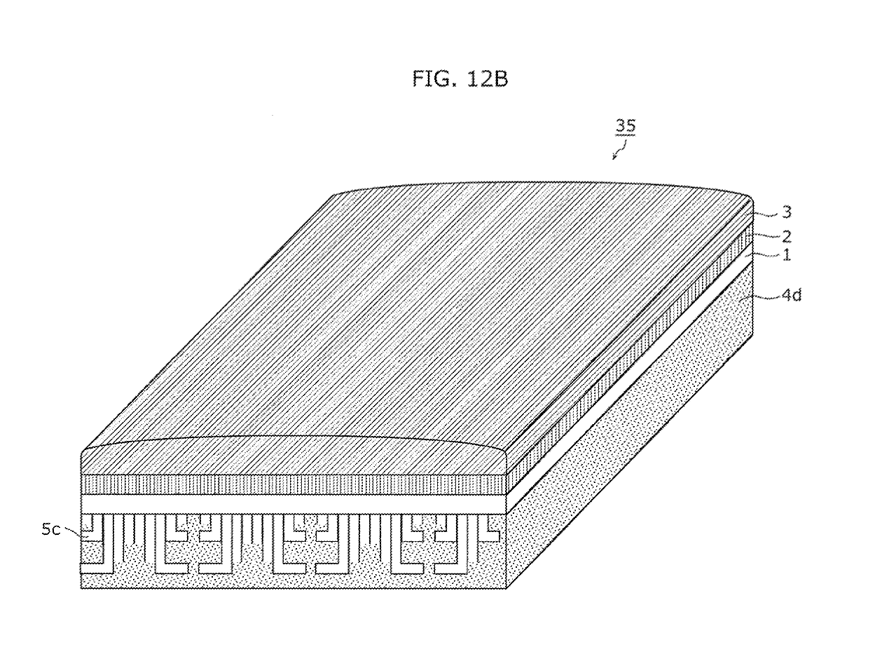

FIG. 12A is a cross-sectional view showing an example of a structure of an ultrasonic transducer according to Embodiment 3 of the present invention.

FIG. 12A shows a specific structure in which an ultrasonic transducer 30 includes the backing layer corresponding to FIG. 10B. The ultrasonic transducer 30 includes a piezoelectric transducer 1 which emits and receives ultrasonic waves, a matching layer 2, an acoustic lens 3, and a backing layer 4c.

The backing layer 4c is provided in contact with the rear of the piezoelectric transducer 1, and attenuates ultrasonic waves emitted in the rear direction from the piezoelectric transducer 1.

The backing layer 4c includes a plurality of reflectors (the acoustic tubes 5) formed in the rear direction from a plane of the backing layer 4c that is in contact with the piezoelectric transducer 1. The reflectors have different lengths based on a principle of superposition of acoustic waves. Here, the reflectors have properties of the acoustic tubes as described above. The following describes the case where the reflectors are the acoustic tubes 5. Stated differently, in the backing layer 4c, the acoustic tubes 5 are arranged and the plane with the openings of the acoustic tubes 5 is provided in contact with the layer of the piezoelectric transducer 1.

The acoustic tubes 5 are formed to have lengths based on the principle of superposition of acoustic waves.

Specifically, each of the acoustic tubes 5 is formed such that its width (w) is sufficiently small compared to the wavelength of the ultrasonic waves emitted from the piezoelectric transducer 1, and its length (Ln) causes direct waves of the ultrasonic waves and reflected waves of the ultrasonic waves to cancel out each other. For example, here, it is assumed that the backing layer 4c is made of an epoxy resin, and the inside of the acoustic tubes 5 is filled with a metal paste that has an acoustic impedance different from an acoustic impedance of the epoxy resin. With this, when it is assumed that the piezoelectric transducer 1 emits 5 MHz ultrasonic waves, wavelength in the acoustic tubes 5 is 600 μm. For example, with an acoustic tube 5 that has a length of 150 μm, a phase of the reflected waves shifts by ¼. This causes cancellation of the ultrasonic waves. Note that the width of the acoustic tube 5 needs to be 150 μm or less because, as described above, the width of the acoustic tube 5 needs to be smaller than the length of the acoustic tube 5. Further, ultrasonic waves having different wavelengths can be cancelled out by arranging in the backing layer 4c the acoustic tubes 5 having different lengths than the above acoustic tube 5. In other words, ultrasonic waves having different frequencies can be cancelled out by arranging in the backing layer 4c the acoustic tubes 5 having different lengths as shown in FIG. 12A.

As described above, when the acoustic tubes 5 are arranged in the backing layer 4c, it is possible to attenuate the ultrasonic waves without increasing the thickness of the backing layer compared to the case where the material having internal loss and distance that can provide attenuation to the ultrasonic waves is provided as the backing layer.

However, when the acoustic tubes are arranged in the backing layer, a thickness of the backing layer needs to be greater than the maximum length of the acoustic tubes. Stated differently, with the ultrasonic transducer according to Embodiment 2, there may be a case where the increase in the thickness of the ultrasonic transducer cannot be sufficiently prevented since the thickness of the backing layer depends on the maximum length of the acoustic tubes.

In view of the above, the following describes an example of a structure with which the increase in the thickness of the backing layer can be prevented even more effectively.

FIG. 12B is a cross-sectional view of another example of a structure of the ultrasonic transducer according to Embodiment 3 of the present invention. In the drawing, the elements identical to the elements shown in FIG. 12A are denoted by the same reference numerals, and thus detailed description thereof are omitted.

The ultrasonic transducer 35 shown in FIG. 12B includes the piezoelectric transducer 1, the matching layer 2, the acoustic lens 3, and a backing layer 4d. In the backing layer 4d, acoustic tubes 5c are arranged and the plane of the backing layer 4d with the openings of the acoustic tubes 5 is provided in contact with the layer of the piezoelectric transducer 1.

The acoustic tubes 5c correspond to the reflectors of the present invention and have lengths based on a principle of superposition of acoustic waves.

Here, the acoustic tubes 5c include at least one acoustic tube 5c which has (i) a portion of the length formed in a direction perpendicular to the rear direction and (ii) the remaining portion of the length formed in a direction parallel to the rear direction. Specifically, each of the acoustic tubes 5c is formed to have a length that is an integer multiple of a predetermined unit length, and at least one of neighboring acoustic tubes 5c which has a greater length has a portion of the length bent in a direction perpendicular to the rear direction so as to be formed in the rear direction of another one of the neighboring acoustic tubes 5c having a smaller length. The neighboring acoustic tubes 5c are included in the acoustic tubes 5c. It is to be noted that the neighboring acoustic tubes 5c are two or more of the acoustic tubes 5c.

More specifically, each of the acoustic tubes 5c is formed such that its width (w) is sufficiently small compared to the wavelength of the ultrasonic waves emitted from the piezoelectric transducer 1, and its length (Ln) causes direct waves of the ultrasonic waves and reflected waves of the ultrasonic waves to cancel out each other. It is to be note that, as shown in FIG. 12B, the acoustic tubes 5c are formed not only in the depth direction of the backing layer 4d but portions of the acoustic tubes 5c are formed in a direction perpendicular to the depth direction of the backing layer 4d. For example, with a length of the acoustic tube 5c having the smallest length among the acoustic tubes 5c as a reference length, a portion of the acoustic tube 5c, which has the length greater than the reference length, may be formed in the direction perpendicular to the depth direction of the backing layer 4d.

Stated differently, except for the acoustic tube having the smallest length, portions of the lengths of the acoustic tubes 5c are bent so as to be formed in a direction perpendicular to the depth direction of the acoustic tube, such that each of the lengths of the acoustic tubes 5 in the depth direction is a sum of (i) the length in the depth direction of the acoustic tube having the smallest length in the depth direction and (ii) the length of width of the corresponding one of the acoustic tubes 5. When a portion of the acoustic tube in the depth direction is formed in the direction perpendicular to the depth direction of the backing layer as described, it is possible to provide the effect of cancelling out the ultrasonic waves, allow the length of the acoustic tube in the depth direction to be small, and further reduce the thickness of the backing layer.

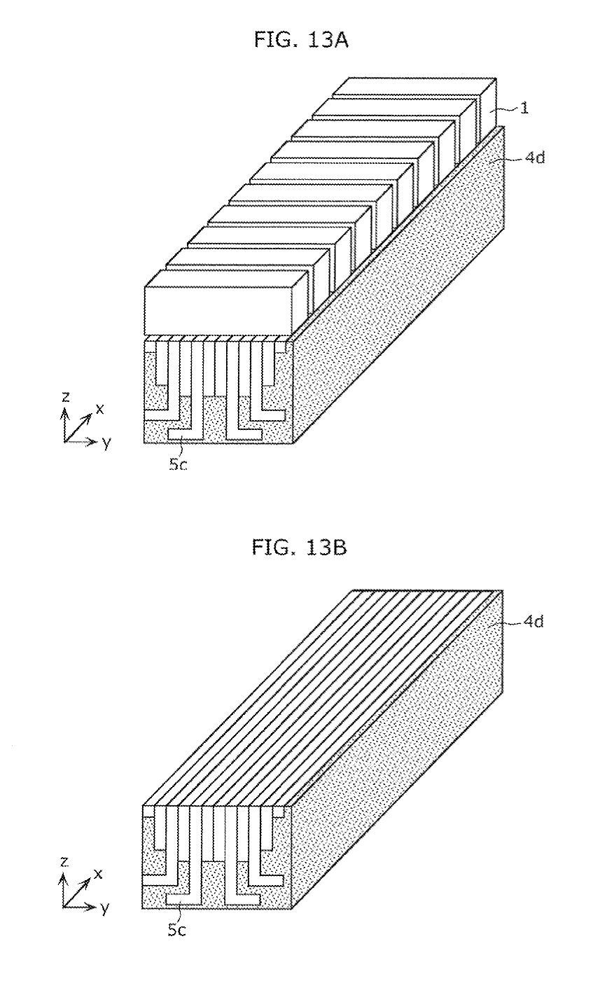

Here, as shown in FIG. 13A, the piezoelectric transducer 1 is sectioned parallel to a short side direction (y direction in the drawing) of the ultrasonic transducer 35 and each of a plurality of channels is independently emits and receives an ultrasonic wave signal. FIG. 13A is a diagram showing an example of an arrangement of the piezoelectric transducer 1 according to Embodiment 3 of the present invention. FIG. 13B is a diagram showing an example of arrangement of the acoustic tubes 5c with respect to the piezoelectric transducer 1 according to Embodiment 3 of the present invention.

Furthermore, as shown in FIG. 13B, the acoustic tubes 5c are arranged such that a cross-section of the ends of openings of the acoustic tubes 5c in contact with the layer of the piezoelectric transducer 1 is parallel to the longitudinal direction (x direction in the drawing) of the ultrasonic transducer 35. In other words, the longitudinal direction (x direction in the drawing) of the cross-section of the ends of openings of the acoustic tubes 5c is substantially perpendicular to the longitudinal direction (y direction in the drawing) of the piezoelectric transducer 1. With such arrangement of the ends of openings of the acoustic tubes 5c, the acoustic tubes 5c having different lengths are arranged for each of the piezoelectric transducer 1, and thus produces an effect that the ultrasonic waves having different frequencies can be canceled out.

It is to be noted that although the structure in which the acoustic tubes 5c are arranged such that the cross-section of the ends of openings of the acoustic tubes 5c are parallel to the longitudinal direction (x direction in the drawing) of the ultrasonic transducer 35, that is, the acoustic tubes 5c are arranged to form grooves has been described, a shape of the cross-section of ends of openings is not limited to this. For example, the cross-section of the ends of openings of each of the acoustic tubes 5c may be formed in a shape of a hole.

Furthermore, the lengths (Ln) of the acoustic tubes 5c are arranged based on a defined rule such as a quadratic residue sequence or a primitive root sequence in the same manner as described in Embodiment 2.

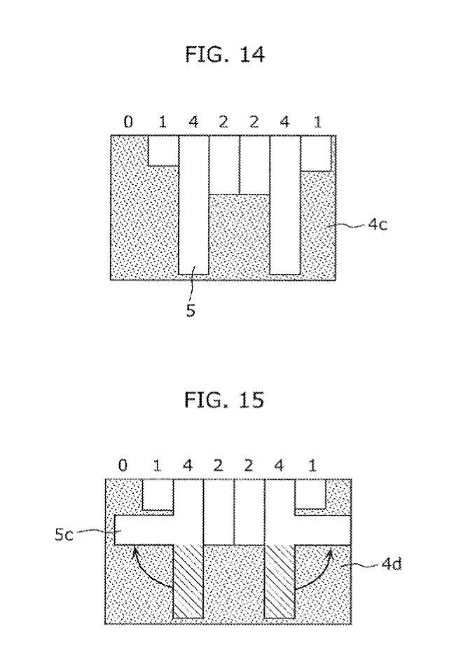

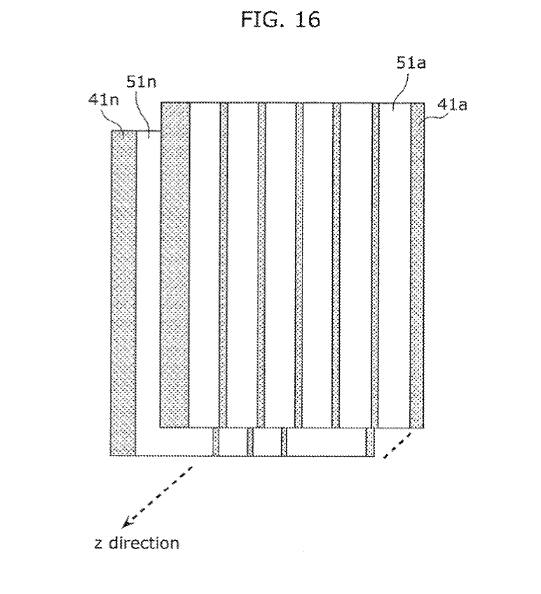

FIG. 14 is a cross-sectional view showing an example of an arrangement of the acoustic tubes 5 shown in FIG. 12A. FIG. 15 is a cross-sectional view showing an example of an arrangement of the acoustic tubes 5c including acoustic tubes which have bent portions shown in FIG. 12B.

For example, it is assumed that length (Ln) of each of the acoustic tubes 5 is arranged based on the quadratic residue sequence indicated by Equation 2. Here, it is assumed that inside of the acoustic tubes 5 is filled with metal paste, speed of sound c=3000 m/s, N=7, and ωr=5 MHz.

In this case, each of the acoustic tubes 5 is arranged, with 43 μm as a unit length “1”, to have a length of 1, 4, 2, 2, 4, 1, and 0, respectively, as shown in FIG. 14.

For example, when the acoustic tubes 5 are arranged as shown in FIG. 14, the acoustic tube 5 having the greatest length needs a length that is four times greater than the acoustic tube 5 having a length equal to a unit length. However, the effect of the acoustic tube does not change even when the acoustic tube is bent along its length. Thus, as shown in FIG. 15, the acoustic tube 5c having a greater length may be bent to a behind of the acoustic tube 5c having a smaller length. With this, it is possible to reduce the thickness of the backing layer 4d as a whole roughly to a half.

As described above, according to the ultrasonic transducer in Embodiment 3, the acoustic tubes having different lengths based on the principle of superposition of acoustic waves are formed in the backing layer in the rear direction (toward lower side in the drawing) from a plane of the backing layer that is in contact with the piezoelectric transducer 1, and, further, portions of the lengths of the acoustic tubes are bent to be formed in a direction perpendicular to the depth direction of the acoustic tube. With this, it is possible to prevent more effectively, the increase in thickness of the backing layer and to attenuate the ultrasonic waves.

### Embodiment 4

Embodiment, 4 describes a manufacturing method that realizes a backing layer according to the present invention.

In other words, Embodiment 4 describes a method of manufacturing an ultrasonic transducer which includes a backing layer that is provided in contact with the rear of a piezoelectric transducer 1. The backing layer includes a board and acoustic tubes, and attenuates ultrasonic waves emitted in the rear direction from the piezoelectric transducer 1.

The following describes an example of a specific process for forming the backing layer which includes a plurality of acoustic tubes (reflectors). The acoustic tubes (reflectors) are formed, by printing on the board (base material) a material with an acoustic impedance different from an acoustic impedance of the board (base material). Each of the acoustic tubes (reflectors) has a different length based on a principle of superposition of acoustic waves, and is formed in the rear direction from a plane of the backing layer that is in contact with the piezoelectric transducer 1. In this process, the acoustic tubes (reflectors) are formed to include at least one acoustic tube which has (i) a portion of the length formed in a direction perpendicular to the rear direction and (ii) the remaining portion of the length formed in a direction parallel to the rear direction.

FIG. 16 is a diagram showing an example of printing patterns according to Embodiment 4 of the present invention. In order to realize the backing layer according to the present invention, a plurality of printing patterns such as the patterns shown in FIG. 16 having relief of 150 μm are formed by screen printing (precision printing). Then, by stacking the formed printing patterns, the backing layers 4d shown in FIG. 12B and FIG. 13B can be manufactured. Stated differently, for example, the printing pattern including a base material 41a and a groove 51a in FIG. 16 is, among layers obtained by dividing the backing layer 4d in a direction perpendicular to a z direction in FIG. 13B, a layer having openings of the acoustic tubes that are in contact with the piezoelectric transducer 1. Furthermore, for example, a printing pattern including a base material 41n and a groove 51n is, among the layers obtained by dividing the backing layer 4d in the direction perpendicular to the z direction in FIG. 13B, the lowermost layer. Then, by adhesively stacking the printing patterns, it is possible to form the backing layer that includes acoustic tubes.

Next, a method of forming the printing patterns shown in FIG. 16 is described.

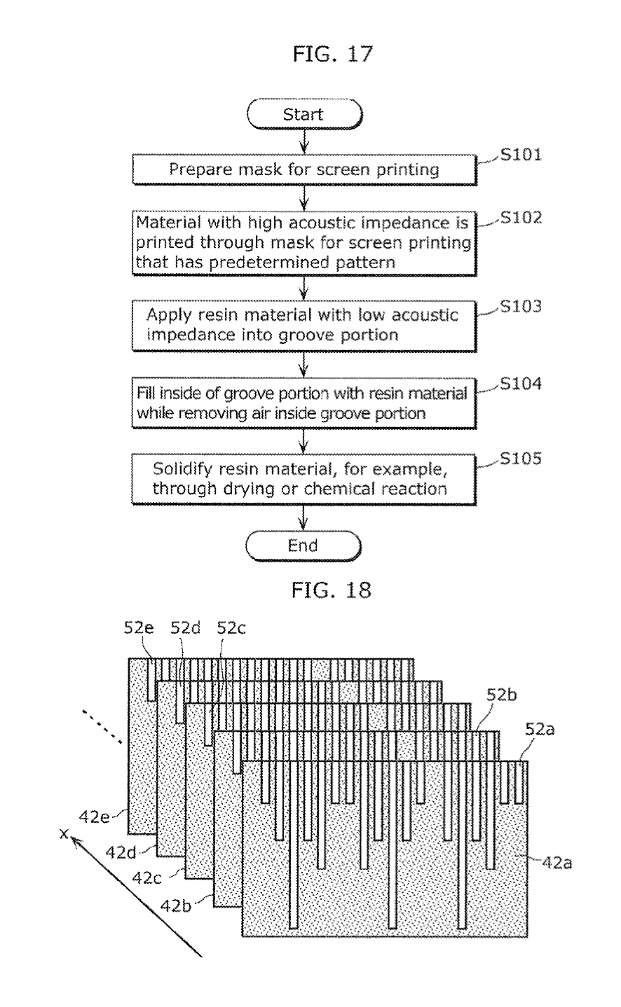

FIG. 17 is a flowchart showing steps for forming a printing pattern using the screen printing according to Embodiment 4 of the present invention.

First, a mask for screen printing that includes groove portion adjusted to have a thickness of 150 μm when dried is prepared (S101).

Next, a material with high acoustic impedance is printed through a mask, which is for screen printing and has a predetermined pattern, such that base material portion is made of a material with high acoustic impedance (S102). Here, the material with high acoustic impedance refers to, for example, metallic conductive paste.

A pattern that forms groove portion of the mask for screen printing needs to be formed such that a bore diameter is equal to or less than 150 μm. With this, a groove having a bore diameter equal to or less than 150 μm can be formed. Thus, the rectilinear propagation of the acoustic waves which enter the groove (the acoustic tube 5c) is good and the ultrasonic waves are reduced in highly effective manner. However, it is not that the effect disappears suddenly once the thickness exceeds 150 μm. Therefore, as far as a desired effect is achieved, the bore diameter does not necessarily have to be exactly 150 μm or less. Note that it is preferable that the base material portion that is formed by printing be made of a material with an acoustic impedance equivalent to or similar to the acoustic impedance of the conductive paste that is used for the printing. With this, reflection of the ultrasonic waves is facilitated.

Next, a resin material with low acoustic impedance is applied into a region on which base material is not present, that is, a groove portion (S103).

Next, a squeegee or the like is used to fill an inside of the groove portion with the resin material while completely removing air inside the groove portion (S104).

Next, the resin material is solidified, for example, through drying or chemical reaction (S105).

Thus, one of the printing patterns shown in FIG. 16 is formed.

Then, by forming the printing patterns shown in FIG. 16 and stacking the formed printing patterns, it is possible to obtain the backing layer 4d which reduces the reflected waves effectively at 5 MHz.

Stated differently, the method of manufacturing the ultrasonic transducer according to this embodiment includes (i) a first process in which base materials (boards) each of which includes a plurality of grooves are formed by printing, (ii) a second process in which the grooves are filled with a material with an acoustic impedance different from an acoustic impedance of the base material by printing, and (iii) a process in which the backing layer 4d which includes the acoustic tubes 5c (reflectors) are formed by adhesively stacking the base materials printed in the first process and the second process.

Thus, by designing the backing layer 4d that includes the acoustic tubes 5c having portions of the lengths bent as shown in FIG. 12B and FIG. 16, the number of printing patterns, i.e. the number of layers stacked, can be reduced compared to the case where the backing layer 4c which includes the acoustic tubes 5 shown in FIG. 12A is designed. In other words, the backing layer which includes acoustic tubes can be manufactured more easily.

It is to be noted that the method of forming the printing patterns shown in FIG. 16 is not limited to the use of the screen printing described above. For example, each of the printing patterns may be formed using a precise mold that is used in, for instance, nanoimprint. In this case, the printing pattern that includes grooves (fine pores) having bore diameters no greater than 150 μm can be formed by embossing against a resin material a mold having a predetermined pattern formed thereon through microfabrication using a nanoimprint technique. Due to the same reason described above, the bore diameter does not necessarily have to be 150 μm or less.

In addition, in the predetermined pattern, the conducting path through which the acoustic waves propagate needs to be formed in a shape of convex. Further, in the same manner as S103 to S105, paste with high acoustic impedance such as metal is applied to the grooves (fine pores) of the obtained printing pattern, and inside the grooves is filled with the paste using a squeegee or the like while completely removing air inside the grooves. Then, the paste is solidified through drying or chemical reaction.

Thus, by forming and stacking the printing patterns shown in FIG. 16, it is possible to manufacture the backing layer which reduces the reflected waves effectively at 5 MHz.

As described above, according to the manufacturing method of the ultrasonic transducer in Embodiment 4, it becomes easier to form the ultrasonic transducer which can attenuate the ultrasonic waves emitted to the rear without increasing the thickness of the backing material.

### Embodiment 5

Embodiment 4 described a method in which printing patterns obtained by dividing a backing layer 4d in a direction perpendicular to a z direction in FIG. 13B are formed to manufacture the backing layer 4d. However, the present invention is not limited to this. Printing patterns obtained by dividing the backing layer 4d in a direction perpendicular to an x direction in FIG. 13B may be formed to manufacture the backing layer 4d.

FIG. 18 is a diagram showing an example of printing patterns according to Embodiment 5 of the present invention.

In this embodiment, in order to realize the backing layer according to the present invention, printing patterns shown in FIG. 18 are formed by screen printing (precision printing), and the formed printing patterns are stacked. Thus, a backing layer 4c shown in FIG. 12A can be manufactured. Stated differently, in FIG. 18, the printing patterns that include a base material 42a and a groove 52a, a base material 42b and a groove 52b, a base material 42c and a groove 52c, a base material 42d and a groove 52d, a base material 42e and a groove 52e . . . , are layers respectively obtained by dividing the backing layer 4c shown in FIG. 12A in a direction perpendicular to the x direction. Then, by stacking these printing patterns, it is possible to form the backing layer 4c that includes acoustic tubes 5.

In other words, the acoustic tubes 5 may be formed not only by stacking the printing patterns in a depth direction (z direction) of the acoustic tubes 5, but also by printing the acoustic tubes 5 divided in the x direction and stacking the printing patterns as shown in FIG. 18.

With this, compared to the method described in Embodiment 4, each of the printing patterns does not have to be accurately stacked. Thus, the backing layer which includes acoustic tubes can be manufactured more easily.

Stated differently, the method of manufacturing the ultrasonic transducer according to this embodiment includes (i) a first process in which base materials (boards) each of which includes a plurality of grooves are formed by printing, (ii) a second process in which the grooves are filled with a material with an acoustic impedance different from an acoustic impedance of the base material by printing, and (iii) a process in which the backing layer 4d which includes the acoustic tubes 5c (reflectors) are formed by stacking the base materials printed in the first process and the second process.

Thus, according to the method of manufacturing the ultrasonic transducer in this embodiment, it is possible to arrange in the backing layer the acoustic tubes which (i) are formed in the rear direction from a plane of the backing layer that is in contact with the piezoelectric transducer, (ii) have different lengths based on the principle of superposition of acoustic waves and, (iii) have portions of the lengths of the acoustic tubes formed in a direction perpendicular to the depth direction of the acoustic tubes.

With this, it is possible to manufacture the ultrasonic transducer that can prevent more effectively the increase in thickness of the backing layer and attenuate the ultrasonic waves.

### Embodiment 6

Embodiment 1 to Embodiment 5 have described the case where the reflector formed in the backing layer, which attenuates ultrasonic waves without increased thickness, is an acoustic tube or a reflector having properties of the acoustic tube. However, the present invention is not limited to these.

As the reflectors corresponding to the acoustic tubes arranged in the backing layer, resonators or reflectors having properties of the resonators may be used. Stated differently, the backing layer which attenuates the ultrasonic waves without increased thickness can also be realized with a resonator that is designed to have a first resonant frequency that is the same as a first resonant frequency of the acoustic tube according to Embodiment 1 to Embodiment 5. Specifically, the backing layer can also be realized with a resonator having a bore diameter and a neck length designed using a Helmholtz resonator principle. With this, it is possible to obtain a similar advantageous effect as the case where the at least one acoustic tube is formed in the backing layer as described in Embodiment 1 to Embodiment 5.

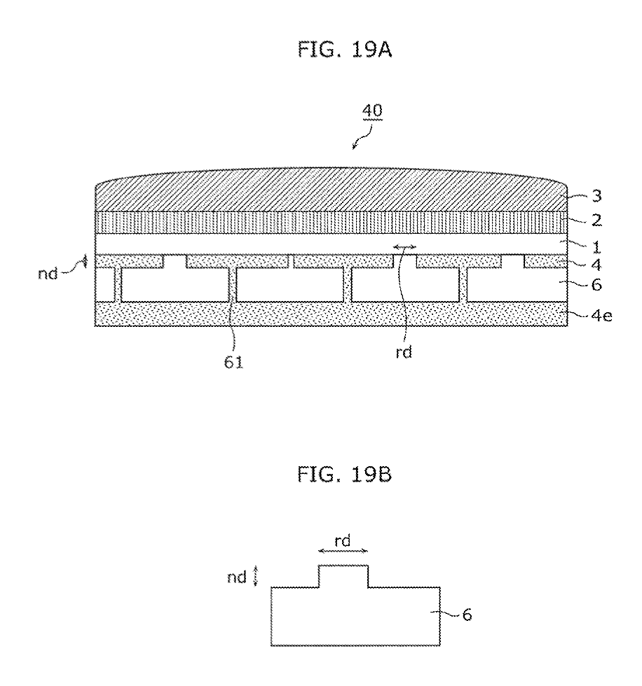

FIG. 19A is a cross-sectional view showing a structure of an ultrasonic transducer according to Embodiment 6 of the present invention. FIG. 19B is a diagram schematically showing a resonator that is an example of the reflector according to Embodiment 6 of the present invention. An ultrasonic transducer 40 shown in FIG. 19A includes a piezoelectric transducer 1 which emits and receives ultrasonic waves, a matching layer 2, an acoustic lens 3, and a backing layer 4e. It is to be noted that, in the drawing, the elements identical to the elements shown in FIG. 12A are denoted by the same reference numerals, and thus detailed description thereof are omitted.

The backing layer 4e is provided in contact with the rear of the piezoelectric transducer 1, and attenuates ultrasonic waves emitted in the rear direction from the piezoelectric transducer 1.

The backing layer 4e includes reflectors (resonators 6) that are formed in the rear direction from a plane of the backing layer 4e that is in contact with the piezoelectric transducer 1. The reflectors (resonators 6) are formed based on the Helmholtz resonator principle. Here, the reflectors have properties of the resonators as described above. The following describes the case where the reflectors are the resonators 6.

Each of the resonators 6 has the neck length and the bore diameter designed to have a desired resonant frequency. Specifically, the resonators 6 can obtain the desired first resonant frequency by designing the bore diameter (rd) and the neck length (nd) shown in FIG. 19B. The first resonant frequency of the resonator 6 can be changed by changing the neck length (nd) and the bore diameter (rd). Thus, it is possible to easily arrange the resonators having various resonant frequencies in the backing layer 4e.

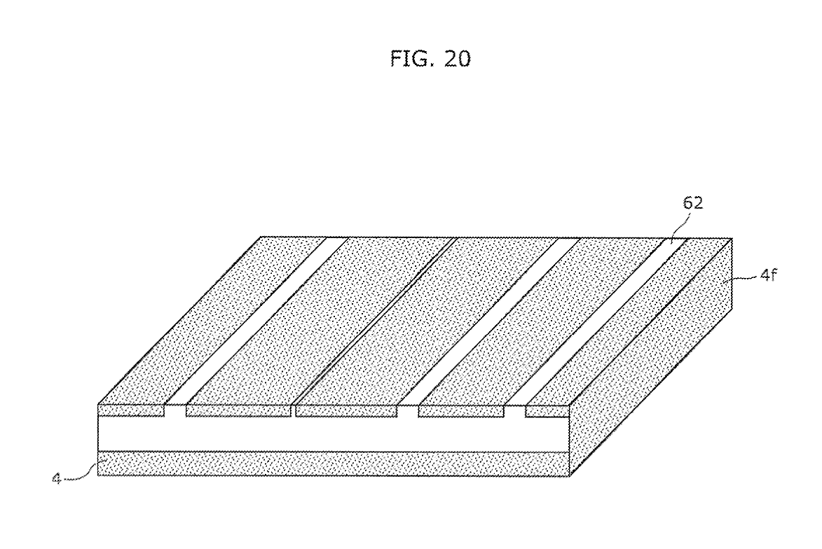

It is to be noted that a distance 61 between the resonators 6 may be any given value. In other words, for example, an inside of the resonator may be connected with the inside of the adjacent resonator as shown in FIG. 20. In this case, a backing layer 4f in which the resonators are arranged can be manufactured more easily. Here, FIG. 20 is a perspective view of the backing layer 4f that shows an example of an arrangement of the resonators 6 according to Embodiment 6 of the present invention.

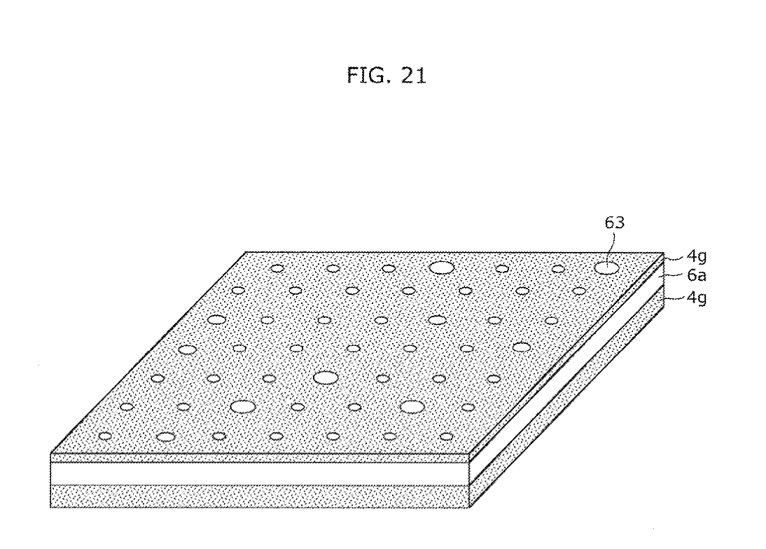

Furthermore, FIG. 21 is a perspective view of a backing layer 4g that shows another example of the resonators according to Embodiment 6 of the present invention. In other words, a shape of the bore portion of the resonator on a plane of the backing layer 4g that is in contact with the piezoelectric transducer 1 may be a slit as shown in FIG. 20 (for example, a slit 62) or may be a hole as shown in FIG. 21 (for example, a hole 63).

Following describes a method of forming the backing layer in which the resonators are arranged as described above. As an example, a method of forming the backing layer 4g shown in FIG. 21 is described.

First, a base material (lower portion of the backing layer 4g in FIG. 21) is formed using metal paste with high acoustic impedance such as silver paste.

Next, on the base material that is formed, a resonator layer (resonator 6a in FIG. 21) is formed using a resin material with small acoustic impedance. Examples of the resin material are rubber polymeric material or plastic such as epoxy, polyester, and polyimide.

Next, a metal layer (upper portion of the backing layer 4g in FIG. 21) that includes holes 63 having different bore diameters is formed on the resonator layer.

Next, the same material as the resonator layer (e.g. resin material) is applied into the holes 63 in the metal layer, and inside of the holes 63 is filled with the material (e.g. resin material) using a squeegee or the like.

Thus, the backing layer which includes resonators shown in FIG. 21 can be formed.

It is to be noted that the base material and the material filled in the holes 63 may be reversed. In other words, a material with high acoustic impedance such as the metal paste may be used to print the structure on the base material made of resin material with small impedance to realize the backing layer.

As described above, according to the ultrasonic transducer in Embodiment 6, the resonators formed in the rear direction from a plane of the backing layer that is in contact with the piezoelectric transducer 1 are arranged in the backing layer. The resonators are formed based on the Helmholtz resonator principle. With this, it is possible to prevent more effectively the increase in thickness of the backing layer and to attenuate the ultrasonic waves.

As described above, according to the present invention, it is possible to provide the ultrasonic transducer and the manufacturing method of the ultrasonic transducer which can attenuate ultrasonic waves emitted to the rear without increasing the thickness of the backing layer.

For example, by arranging as reflectors the acoustic tubes or the resonators in the backing layer, it is possible to attenuate the reflected waves in the backing layer 4 and increase sensitivity of the ultrasonic transducer.

Further, heat can be released to outside of the backing layer using the acoustic tubes or the resonators, and thus there is an effect that the heat contained in the backing layer can be dissipated.

Although the ultrasonic transducer and the manufacturing method of the ultrasonic transducer according to the present invention have been described thus far based on the above embodiments, the present invention is not limited to such embodiments. The scope of the present invention includes embodiments obtained through various modifications to the above embodiments or embodiments obtained through a combination of elements of above embodiments that may be conceived by those skilled in the art without departing from the spirit of the present invention.

For example, ultrasonic diagnostic apparatuses which use the ultrasonic transducers according to the present invention are intended to be included within the scope of the present invention.

### INDUSTRIAL APPLICABILITY

The present invention is applicable to, for example, ultrasonic transducers used by ultrasonic diagnostic apparatuses and methods for manufacturing the ultrasonic transducers. The present invention is particularly useful in realizing an ultrasonic transducer and a method of manufacturing the ultrasonic transducer which reduce reflected waves in a backing layer, increase sensitivity of a received ultrasonic wave signal, reduce thickness of the ultrasonic transducer, and reduce cost of manufacturing as a result of the thinner ultrasonic transducer.

What is claimed is:

1. An ultrasonic transducer comprising:
a transducer which emits and receives ultrasonic waves; and
a backing material which is provided in contact with a rear of said transducer and which attenuates the ultrasonic waves emitted in a rear direction from said transducer,
wherein said backing material includes a plurality of reflectors formed in the rear direction from a plane of said backing material that is in contact with said transducer, each of said reflectors having a different length based on a principle of superposition of acoustic waves, and
said reflectors include a reflector which has (i) a portion of the length formed in a direction perpendicular to the rear direction and (ii) the remaining portion of the length formed in a direction parallel to the rear direction.

2. The ultrasonic transducer according to claim 1,
wherein each of said reflectors has properties of an acoustic tube.

3. The ultrasonic transducer according to claim 1,
wherein each of said reflectors is formed to have a length that is an integer multiple of a predetermined unit length, and
one of neighboring reflectors which has a greater length has a portion of the length bent in a direction perpendicular to the rear direction so as to be formed in the rear direction of another one of said neighboring reflectors having a smaller length, said neighboring reflectors being included in said reflectors.

4. An ultrasonic transducer comprising:
a transducer which emits and receives ultrasonic waves; and
a backing material which is provided in contact with a rear of said transducer and which attenuates ultrasonic waves emitted in a rear direction from said transducer,
wherein said backing material includes a plurality of reflectors formed in the rear direction from a plane of said backing material that is in contact with said transducer, each of said reflectors being formed based on a Helmholtz resonator principle.

5. The ultrasonic transducer according to claim 4,
wherein said reflectors have properties of the resonator, and
each of said reflectors has a neck length and a bore diameter that are designed to have a desired resonant frequency.

6. The ultrasonic transducer according to claim 1,
wherein said backing material includes
a board and said reflectors, and
said reflectors are made of a material with an acoustic impedance different from an acoustic impedance of said board.

7. The ultrasonic transducer according to claim 6,
wherein said reflectors are formed on said board by printing.

8. A method of manufacturing an ultrasonic transducer which includes: a transducer which emits and receives ultrasonic waves; and
a backing material which (i) is provided in contact with a rear of the transducer, (ii) includes a board and a plurality of reflectors, and (iii) attenuates ultrasonic waves emitted in a rear direction from the transducer, said method comprising
forming the backing material which includes the reflectors by printing on the board a material with an acoustic impedance different from an acoustic impedance of the board, each of the reflectors having a different length based on a principle of superposition of acoustic waves and being formed in the rear direction from a plane of the backing material that is in contact with the transducer.

9. The method of manufacturing an ultrasonic transducer according to claim 8,
wherein in said forming of the backing material, the reflectors are formed to include a reflector which has (i) a portion of the length formed in a direction perpendicular to the rear direction and (ii) the remaining portion of the length formed in a direction parallel to the rear direction.

10. The method of manufacturing an ultrasonic transducer according to claim 8,
wherein said forming of the backing material includes:
forming, by printing, base materials each of which includes a plurality of grooves;
filling the grooves, by printing, with a material with an acoustic impedance different from an acoustic impedance of the base material; and
forming the backing material which includes the reflectors, by adhesively stacking the base materials printed in said forming of the base material and in said filling of the grooves.

11. The method of manufacturing an ultrasonic transducer according to claim 10,
wherein in said forming of the base material, the base materials each of which includes a plurality of grooves which have different lengths based on the principle of superposition of acoustic waves are formed by printing.

12. An ultrasonic diagnostic apparatus comprising the ultrasonic transducer according to claim 1.
